(19) United States
(12) Patent Application Publication
(10) Pub. No.: US 2009/0298166 A1
(43) Pub. Date: Dec. 3, 2009

(54) CELL CULTURE APPARATUS HAVING VARIABLE TOPOGRAPHY

(76) Inventors: Ye Fang, Painted Post, NY (US); Jianguo Wang, Horseheads, NY (US); Ying Zhang, Corning, NY (US)

Correspondence Address: CORNING INCORPORATED, SP-TI-3-1, CORNING, NY, 14831, US

(21) Appl. No.: 12/471,879

(22) Filed: May 26, 2009

Related U.S. Application Data
(60) Provisional application No. 61/130,364, filed on May 30, 2008.

(51) Int. Cl. C12M 1/00; B44C 1/22

(52) U.S. Cl. 435/305.2; 216/41

(57) ABSTRACT

A cell culture apparatus includes a substrate having formed therein a micro-pillared well array. The micro-pillared well array includes a plurality of micro-pillared wells. Each micro-pillared structure includes a plurality of spaced-apart micro-pillars having distal ends shaped to form a well. The well is suitable for cell culture.

### CROSS REFERENCE TO RELATED APPLICATION

This Application claims the benefit of U.S. Provisional Application Ser. No. 61/130,364 filed May 30, 2008 and entitled “Cell Culture Apparatus Having Variable Topography”.

### FIELD

The invention relates generally to apparatus for cultivating biological cells. More specifically, the invention relates to an apparatus for cultivating three-dimensional, multicellular clusters.

### BACKGROUND

Traditionally, in-vitro models for biomedical studies are based on two-dimensional cell cultures, where cells are cultured on a planar surface. However, despite the experimental convenience and good cell viabilities of two-dimensional cell cultures, it has been commonly acknowledged that cell behaviors, such as phenotypes, function, and regulation of signaling pathways, can be fundamentally different between a two-dimensional cell layer and a complex three-dimensional multicellular cluster. A good example is a cancer study where researchers found that three-dimensional malignant breast tumor cells can revert back to their original states with addition of a certain antibody—this curing effect had never been observed in two-dimensional culture. (Bin Kim J., Stein R. and O'Hare M. J., “Three-dimensional in vitro tissue culture models of breast cancer—a review,” Breast Cancer Research and Treatment 2004, 85(3):281-291.) Because the three-dimensional culture systems share more similarities with the physiological environments found in the living organism, the development of a more effective in-vivo three-dimensional cell culture system would be critical for many research areas, such as new drug development, stem and cancer research, and tissue engineering.

The essential environment of in-vivo cells is the existence of extracellular matrix, which provides cell-substrate interaction support and facilitates nutrition and metabolic waste transportation. Research on cell-substrate interactions have shown that for a specific substrate surface, different cells can show significantly different behaviors in terms of, for example, cell adhesion, morphology, orientation, mobility, and bioactivities. The ultimate aim of cell-substrate interaction research is thus to obtain the optimum substrate surface for directing cell culture and development. However, much of the present research dwells on obtaining three-dimensional cell clusters at defined locations by physically or chemically confining cell growth. In physical confinement, an array of wells is fabricated. Subsequently, cell culture is restricted within each micro-well to form multicellular clusters. Typically, the wells have steep sidewalls and a planar bottom so that even with confinement the cells grow on a planar surface. Thus, the cells do not interact with a true three-dimensional surface.

### SUMMARY

In one aspect, the invention relates to a cell culture apparatus which comprises a substrate having formed therein micro-pillared cell culture wells. In embodiments, these micro-pillared cell culture wells maybe in a micro-pillared well array. The micro-pillared well array comprises a plurality of micro-pillared well structures. Each micro-pillared well structure comprises a plurality of spaced-apart micro-pillars having distal ends shaped to form a well.

In another aspect, the invention provides a cell culture apparatus, formed from a substrate having a plurality of micro-wells wherein the micro-wells are formed from a plurality of micro-pillars. In embodiments of the invention, the micro-wells are grouped to form a plurality of micro-well arrays. The micro-well arrays may contain micro-wells that have the same topography, or the micro-well arrays may contain micro-wells that have different or variable topography. In further embodiments, the micro-well arrays may have at least one micro-channel formed in the substrate between adjacent micro-well arrays to allow fluid to flow between arrays. In still further embodiments, the micro-wells may be recessed relative to a surface of the substrate.

In another aspect, the invention relates to a method of making a cell culture apparatus which comprises forming a plurality of microdots on a first substrate, each microdot being a negative copy of a well. The method includes forming a plurality of micro-pillars on the plurality of microdots so that the micro-pillars abut the plurality of microdots. The number of micro-pillars is substantially larger than the number of microdots so that a plurality of the micro-pillars abut each microdot. The method includes transferring intersecting profiles of the microdots and micro-pillars to a second substrate to form a plurality of micro-pillared structures in the second substrate.

Other features and advantages of the invention will be apparent from the following description and the appended claims.

### BRIEF DESCRIPTION OF DRAWINGS

The accompanying drawings, described below, illustrate typical embodiments of the invention and are not to be considered limiting of the scope of the invention, for the invention may admit to other equally effective embodiments. The figures are not necessarily to scale, and certain features and certain views of the figures may be shown exaggerated in scale or in schematic in the interest of clarity and conciseness.

FIG. 1 is a perspective view of a cell culture apparatus having a variable topography.

FIGS. 2A-2C depict vertical slices of well geometries for inclusion in a cell culture apparatus having variable topography.

FIG. 3 is a vertical slice of the cell culture apparatus of FIG. 1.

FIG. 4 shows the vertical slice of FIG. 3 with recessed micro-pillared structures.

FIG. 5 shows the vertical slice of FIG. 3 with micro-channels formed between adjacent micro-pillared wells.

FIG. 6 is a top view of a cell culture apparatus having a plurality of micro-pillared well arrays.

FIG. 7 is a cross-sectional view of a cell culture apparatus having micro-channels between adjacent micro-pillared well arrays.

FIG. 8A illustrates photo-patterning of a substrate

FIG. 8B shows the photo-patterned substrate of FIG. 8A after etching.

FIG. 8C shows the substrate of FIG. 8B after a reflow process.

FIG. 8D shows photo-patterning of the substrate of FIG. 8C.

FIG. 8E shows micro-pillars being formed in the substrate of FIG. 8D.

FIG. 8F shows micro-pillars and microdots molded into a substrate.

FIG. 8G shows a substrate with micro-pillared wells.

FIG. 8H shows metal grown over the substrate of FIG. 8G.

FIG. 8I shows a replication mold formed from the grown metal of FIG. 8H.

### DESCRIPTION OF EMBODIMENTS

The invention will now be described in detail with reference to a few preferred embodiments, as illustrated in the accompanying drawings. In describing the preferred embodiments, numerous specific details are set forth in order to provide a thorough understanding of the invention. However, it will be apparent to one skilled in the art that the invention may be practiced without some or all of these specific details. In other instances, well-known features and/or process steps have not been described in detail so as not to unnecessarily obscure the invention. In addition, like or identical reference numerals are used to identify common or similar elements.

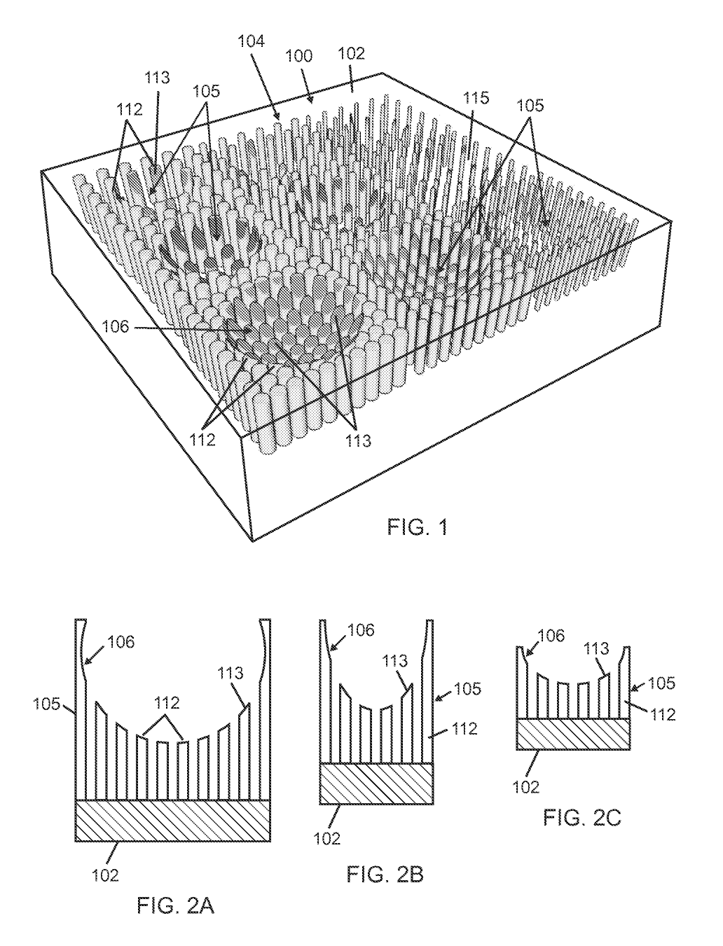

FIG. 1 depicts a cell culture apparatus 100 including a substrate 102, which may be made of any biocompatible material suitable for cultivating biological cells. For example, the biocompatible material could be a biocompatible polymer, such as polydimethylsiloxane, polyethylene, polystyrene, polyolefin, polyolefin copolymers, polycarbonate, ethylene vinyl acetate, polypropylene, polysulfone, polytetrafluoroethylene (PTFE) or compatible fluoropolymer, a silicone rubber or copolymer, poly(styrene-butadiene-styrene), acrylic, or polyester or combinations of these materials. Other examples of suitable materials include glass, quartz, gold, silicon dioxide, and titanium-coated silicon. An array of micro-pillared wells 104 is formed in the substrate 102. The array of micro-pillared wells 104 includes a plurality of micro-pillared structures or wells 105. Each micro-pillared structure or well 105 includes a plurality of spaced-apart micro-pillars 112 having distal ends 113 shaped to form a depression or concavity or receptacle or well 106 for receiving and culturing biological cells. For example, the micro-pillared structure 105 has micro-pillars 112. Some of the micro-pillars are shaped to form a well 106, a recessed or depressed area structured and arranged to contain cells in cell culture. The micro-pillars 112 may have a variety of cross-sectional shapes, such as circular, elliptical, triangular, square, and rectangular. The substrate material used to fabricate the micro-pillars 112 tailors the mechanical properties of the micro-pillars 112 and the cell culture apparatus 100. In the embodiment shown in FIG. 1, the micro-pillars 112 have a circular cross-section. The micro-pillars 112 may be arranged in the substrate 102 using a variety of packing geometries, such as hexagonal or cubic packing geometry. The wells 106 may have a variety of shapes. In embodiments, the wells may be, for example, cylindrical with a flat bottom surface, cuboid with a flat bottom surface, pyramidal with a pointed bottom, conical with a pointed bottom or truncated conical or pyramidal with a flat bottom surface. In one embodiment, the wells 106 are curved. In another embodiment, the wells 106 are concave. In one embodiment, the concave shape of the wells is an elliptic paraboloid. The term “elliptic paraboloid” also includes the special case of a circular paraboloid, as illustrated in FIG. 1. FIGS. 2A-2C show additional examples of concave wells 106. Each of the micro-pillared wells shown in FIGS. 2A-2C have different curvature K. Shaped wells, such as concave wells, may enhance formation of three-dimensional cell spheroids in a variety of sizes and morphologies. FIG. 2 illustrates three wells 106 having different or variable topography, for example, the three wells shown in FIGS. 2A-2C have different curvature K, different height h and different diameter d.

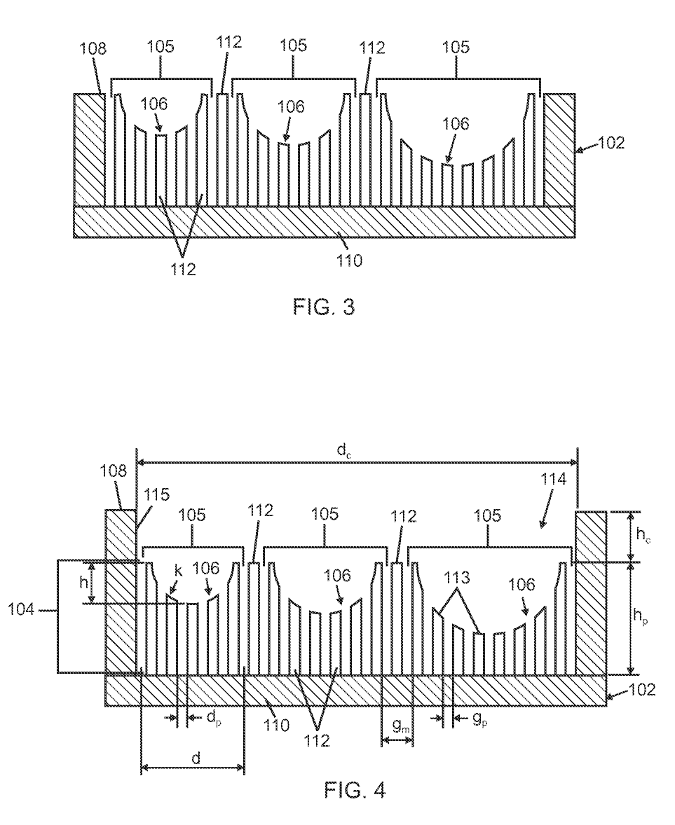

FIG. 3 shows a slice of the cell culture apparatus 100. The micro-pillared structures 105 extend from the top surface 108 of the substrate 102 to a base portion 110 of the substrate 102. In this case, the wells 106 are open to the surface 108 of the substrate 102. In an alternate embodiment, as shown in FIG. 4, the micro-pillared structures 105 are recessed relative to the top surface 108 of the substrate 102, resulting in a macro-well or chamber 114 above the array of micro-pillared structures 104. The chamber 114 has a side wall 115, which in FIG. 4 is cylindrical. In alternate embodiments, the chamber 114 may have other shapes, such as an inverted, truncated cone shape or a parallelepiped shape, with or without rounded corners. The depth (h_{c}) of the chamber 114 determines how far the micro-pillared structure array 105 is recessed relative to the top surface 108 of the substrate 102.

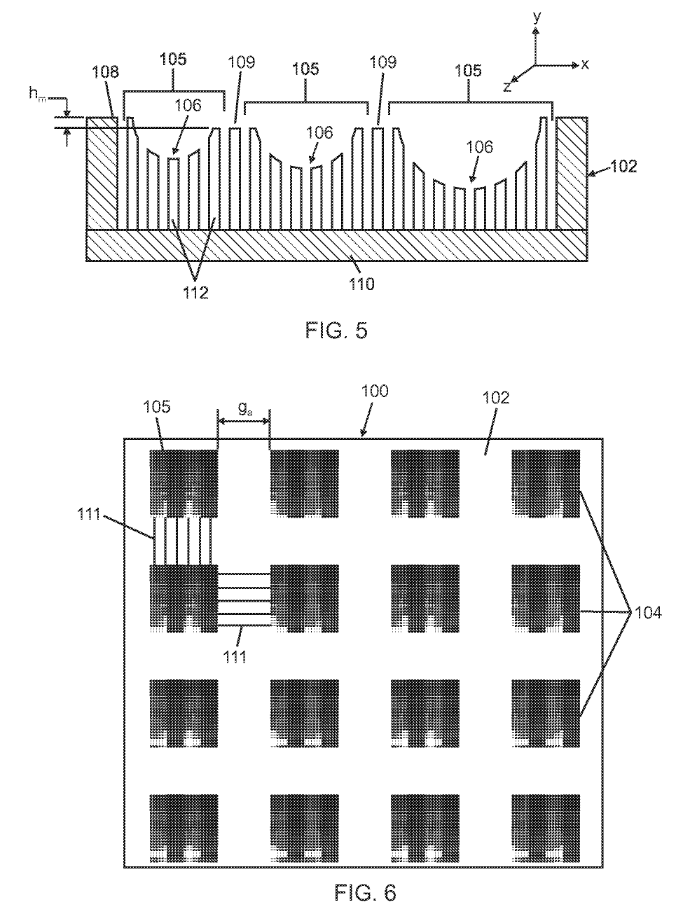

In another embodiment, as illustrated in FIG. 5, adjacent wells 106 may be interconnected via micro-channels 109. These micro-channels 109 may be formed by truncating one or more of the micro-pillars 112 between adjacent micro-pillared structures 105. The micro-channels 109 can promote communication between cell clusters in adjacent wells 106 and also enable cells located within the wells 106 to have free access to cell culture medium e.g., growth factors or stimulus, within the micro-pillared structure array 105. The width of the micro-channel 109 is typically determined by the width of the wells 106 it interconnects. In general, the width of each micro-channel 109 (measured along the z-axis) may range from 5 to 50 microns. The depth (hₘ) of each micro-channel 109 can be flexible. In general, the depth of each micro-channel 109 is not greater than the depth of the wells 106 that it interconnects. The recessed micro-pillared structure array 104 shown in FIG. 4 may also include interconnecting micro-channels similar to the micro-channels 109 shown in FIG. 5.

Referring to FIG. 4, each micro-pillared structure 105 has a topography which is described by the following micro-pillared structure parameters: the diameter (d) of the well 106, the height (h) of the well 106, the curvature (k) of the well 106, the diameter (dₚ) of the micro-pillars 112 defining the well 106, the spacing or gap (gₚ) between the micro-pillars 112 defining the well 106, the height (hₚ) of each micro-pillar 112 defining the well 106, the packing geometry of the micro-pillars 112 defining the well 106, any surface treatment on the distal ends 113 of the micro-pillars 112 defining the well 106, and cross-sectional shape of the micro-pillars 112 defining the well 106. The spacing (gₚ) between the micro-pillars 112 is determined by the size of the biological cells to be cultured in the wells 106. Typically, the spacing (gₚ) between the micro-pillars 112 is smaller than the biological cells such that after culture the cells remain within the wells 106 and have limited or no penetration into the spaces between the micro-pillars 112. In a preferred embodiment, the diameter (d) of each well 106 ranges from 50 to 500 microns. In a preferred embodiment, the height (h) of each well 106 ranges from 0 to 100 microns. At 0 microns, the well 106 is flat. In an embodiment, the height of each well 106 ranges from 5 to 100 microns. In another embodiment, each micro-pillar 112 has a diameter ranging from 0.5 to 20 microns. In another embodiment, the spacing (gₚ) between adjacent micro-pillars 112 is in a range from 5 to 100 microns. The micro-pillared structure array 104 has a topography that is described by the individual topographies of all the micro-pillared structures 105 within the array and the following array parameters: spacing (gₘ) between adjacent micro-pillared structures 105, height (h_{c}) of the chamber 114 if present, diameter (h_{c}) of the chamber 114 if present, and shape of the chamber 114 if present. The spacing (gₘ) between adjacent micro-pillared structures 105 preferably ranges from 10 to 500 microns.

FIG. 1 shows the cell culture apparatus 100 as having a single array of micro-pillared structures 104 with nine micro-pillared well structures 105. In practice, the cell culture apparatus 100 can have several more micro-pillared structures 105 arranged in a plurality of micro-pillared structure arrays 104. In FIG. 6, for example, the cell culture apparatus 100 includes a plurality of micro-pillared structure arrays 104, wherein each micro-pillared structure array 104 includes a plurality of micro-pillared structures 105. Because of the scale of the drawing, the micro-pillared structures 105 appear as dots in this figure. In general, the micro-pillared structure array 104 would resemble the one shown in FIG. 1, except that in FIG. 6 there are more micro-pillared structures 105 which may have different topographies from that shown in FIG. 1. In FIG. 6, the micro-pillared structure arrays 104 may have equal number or different numbers of micro-pillared structures 105. In FIG. 6, each micro-pillared structure array 104 has a quadrate shape. In alternate embodiments, the micro-pillared structures 105 may be arranged such that the micro-pillared structure array 104 has a non-quadrate shape, such as a circular or hexagonal shape. Any of the micro-pillared structure arrays 104 in FIG. 6 may be a recessed micro-pillared structure array 104 as described with respect to FIG. 4.

Referring to FIG. 6, the micro-pillared structure arrays 104 are spaced apart within the substrate 102. The spacing (gₐ) between the micro-pillared structure arrays 104 may be uniform or non-uniform across the substrate 102. Any suitable arrangement of the micro-pillared structure arrays 104 in the substrate 102 may be used, but it may be convenient to arrange the arrays of micro-pillared structures 104 as an N×M rectangular or square array, where N>1, M>1. In one example, N×M has a value selected from 6, 12, 16, 24, 96, and 384. In the example shown in FIG. 4, N×M is 16.

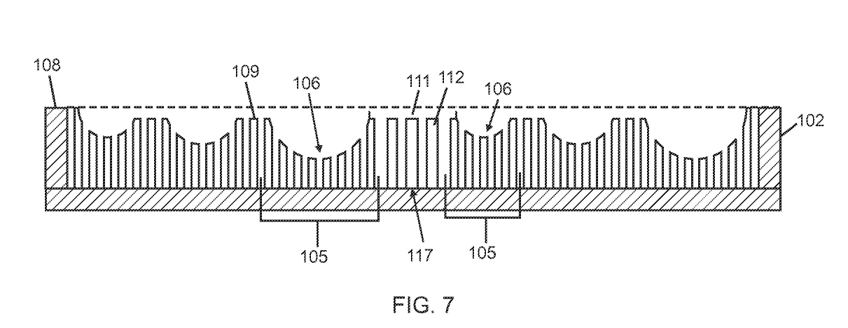

One or more micro-channels may be formed between adjacent micro-pillared structure arrays 104 in the cell culture apparatus 100. This is suggested by lines 111 in FIG. 6 and illustrated more fully in FIG. 7 for two adjacent micro-pillared structure arrays 104. In FIG. 7, one or more micro-channels 111 are formed between the adjacent micro-pillared structure arrays 104. If the micro-pillared structure arrays 104 were viewed from the top, the micro-channels 111 would appear as channels or grooves straddling between the micro-pillared structure arrays 104. The micro-channels 111 interconnect the wells 106 of the micro-pillared structures 105 in the adjacent micro-pillared structure arrays 104. The interconnecting micro-channels 111 can also be used where the micro-pillared structure arrays 104 are recessed relative to the top surface 108 of the substrate 102 as previously described. The micro-channels 111 enable cells located within the wells 106 to have free access to cell culture medium, e.g., growth factors or stimulus, within the cell culture apparatus 100. The micro-channels 111 can also promote communication between two cell clusters located in wells 106 in adjacent micro-pillared structure arrays 104. The width of the micro-channel 111 is typically determined by the width of the wells 106 it interconnects. In general, the width of the micro-channels 111 may range from 5 to 50 microns. The depth of the micro-channels 111 can be flexible. In general, the depth of the micro-channel 111 is not greater than the depth of the wells 106 it interconnects. FIG. 7 also shows that the region 117 of the substrate 102 between adjacent micro-pillared structure arrays 104 may include micro-pillars 112, i.e., instead of being completely solid.

Referring to FIGS. 1-7, the cell culture apparatus 100 may be configured as a screening tool for identifying optimum micro-environments for culturing of specific biological cells. Where the cell culture apparatus 100 is configured as a screening tool, at least two, a plurality, of the micro-pillared structures 105 may have different topographies. Two micro-pillared structures 105 have different topographies if at least one of the aforementioned micro-pillared structure parameters are different in value. For example, two micro-pillared structures 105 or micro-wells 106 may have different or variable topographies if they have different shapes, different curvature, different height, depth, spacing between wells, or different surface treatment. The at least two micro-pillared structures 105 having different topographies may be located within a single array 104 or in two different arrays 104. For example, a cell culture device may have a plurality of arrays of micro-pillared wells where all of the micro-pillared wells in all of the arrays of micro-pillared wells have the same topography, or are uniform. Or, a cell culture device may have a plurality of arrays of micro-pillared wells where the micro-pillared wells within each array are heterogeneous, or non-uniform, or variable, or have different topography, as shown in FIG. 6. Or, a cell culture device may have a plurality of arrays where the wells within each array are uniform, but the wells of one array may be different from the wells of another array.

When the cell culture apparatus 100 is configured for a specific cell line, the micro-pillared structures 105 in the cell culture apparatus 100 may have the same topography. For co-culturing of a plurality of cell lines, the cell culture apparatus 100 may include a plurality of micro-pillared structure arrays 104, wherein each micro-pillared structure array 104 includes micro-pillared structures 105 tailored to a specific cell line. In this case, the micro-pillared structures 105 within each micro-pillared structure array 104 may also have the same topography. Earlier on, it was mentioned that each micro-pillared structure has a topography and that each micro-pillared structure array has a topography. The cell culture apparatus 100 also has a topography which is described by the collective topographies of the micro-pillared structure arrays 104 it contains and the spacing between the micro-pillared structure arrays 104.

The cell culture apparatus 100 described above in FIGS. 1-7 can be used to cultivate three-dimensional multicellular clusters. The wells 106 define the shape and diameter of the cell cluster while the micro-pillars 112 control the cell-substrate interaction. The wells 106 and micro-pillars 112 can mimic the essential environment of in-vivo cells and facilitate nutrition and metabolic waste transportation. As a screening tool, the cell culture apparatus 100 can facilitate discovery of optimum or effective microenvironment for three-dimensional cell culture of different types in that the influence of microenvironment on cell culture can be rapidly and effectively studied by comparing a broad range of cell culture results on a single chip. In particular, the cell culture apparatus 100 makes it easy to investigate the effect of one or more micro-pillared structure parameters on the cell culture microenvironment on a single chip. The location of each micro-pillared structure 105 in the substrate 102 and the arrangement of the micro-pillared structures 105 in the substrate 102 are known. This information can be stored in any suitable format for later use in identifying the micro-pillared structure 105 (or micro-pillared structure array 104) that behave optimally or effectively in terms of cell-substrate interaction for a particular cell type. In one example, the location and description of the micro-pillared structures 105 are stored in a structured file, such as an XML file, or other computer-readable structured format. The stored location and description of the micro-pillared structures 105 are then used to create an initial digital image of the cell culture apparatus 100. After initiation of a cell-substrate interaction study, subsequent digital images of the cell culture apparatus 100 are generated. These subsequent digital images may be compared to the initial image to assist in identifying micro-pillared structures 105 (or micro-pillared structure arrays 104) that behave optimally.

For completeness, an example of a method of making the cell culture apparatus described above is presented herein. However, the cell culture apparatus described above could be made via any suitable process or combination of processes known in the art.

In one example, a method of making the cell culture apparatus as described above includes forming a plurality of microdots on a first substrate. Each microdot is a negative copy of a well as described above. The plurality of microdots may be formed on the first substrate by any suitable process such as casting or photolithography, followed by resist reflow. A plurality of micro-pillars are formed on the plurality of microdots such that the micro-pillars abut the plurality of microdots. The abutting profiles of the microdots and micro-pillars are then transferred to a second substrate to form a plurality of micro-pillared structures in the second substrate. The process may include further steps where the cell culture apparatus includes chambers formed above the micro-pillared structures.

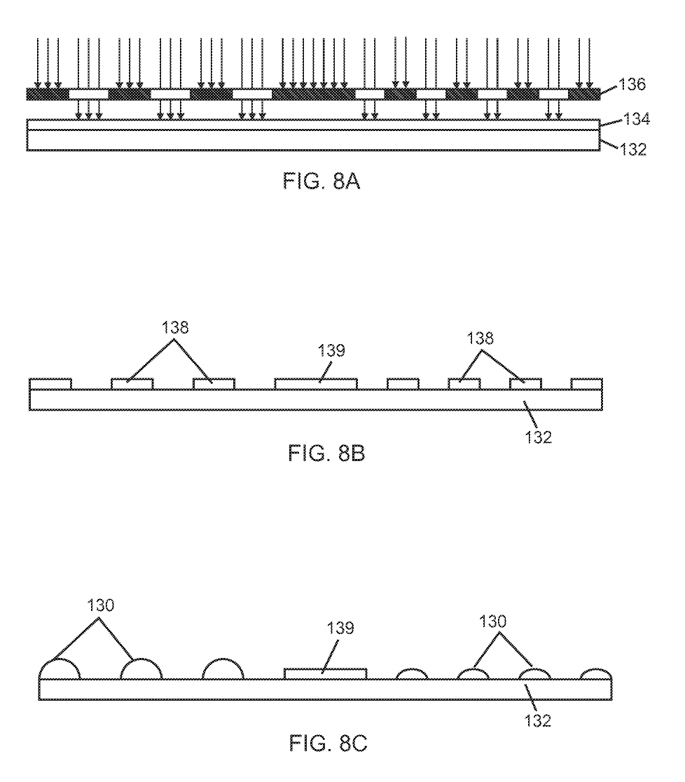

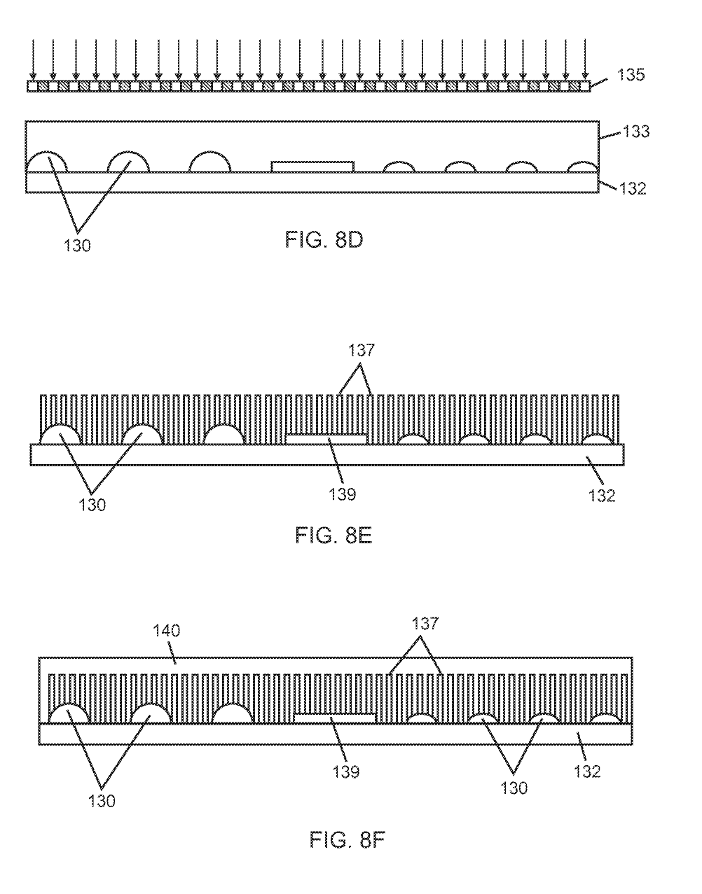

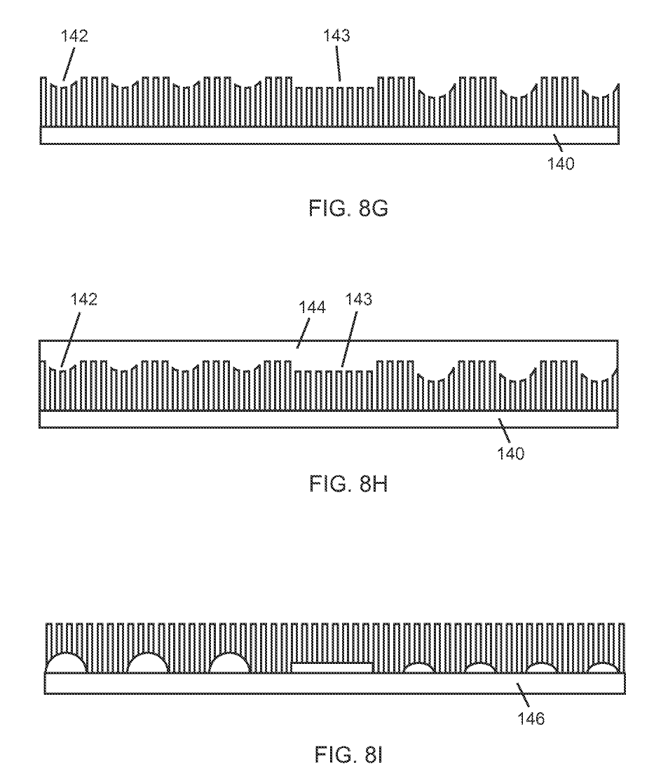

In FIGS. 8A and 8B, a method of making the plurality of microdot arrays by photolithography, followed by resist reflow, is described. In FIG. 8A, a photoresist 134 is deposited on a first substrate 132 using any suitable process known in the art, such as spin coating. The first substrate 132 may be made of any suitable substrate material, such as glass, polymer, and silicon. Preferably, the first substrate 132 has a hydrophilic surface to facilitate adhesion to the photoresist 134. Preferably, the photoresist 134 is a positive photoresist. The thickness of the photoresist 134 on the first substrate 132 is generally determined by the final depth of the wells to be formed. Next, the photoresist 134 is exposed to a pattern of light through a photomask 136. The pattern of light is determined by the configuration and arrangement of the microdots to be formed. The exposed photoresist 134 is developed and etched to form a plurality of micro-posts. FIG. 8B shows the micro-posts 138. It should be noted that to avoid crowding the figure, only a few micro-posts 138 from two adjacent arrays of micro-pillars are shown. A micro-channel post 139 is formed between the two adjacent arrays of microdots. The micro-channel post 139 may be made from material having a higher melting temperature than the micro-post material. The micro-posts 138 in FIG. 8B are shaped into microdots 130 in FIG. 8C by a resist reflow process. As previously mentioned, the microdots are negative copies of the wells to be formed. The resist reflow process includes heating the micro-posts 138 in FIG. 8B above the glass transition temperature of the first substrate material, wherein the micro-posts 138 deform by surface tension to form the microdots 130 in FIG. 8C. The shape of the microdots 130 in FIG. 8C is governed by the height and diameter of the micro-posts 138 in FIG. 8B and controlled by the degree and duration of the heat applied to the micro-posts.

Referring to FIG. 8D, a method of forming micro-pillars on the microdots 130 and micro-channel post 139 includes depositing photoresist 133 on the microdots 130, micro-channel post 139, and first substrate 132 using any suitable process known in the art, such as spin coating. Any suitable photo-resist material may be used. The photoresist 133 is exposed to a pattern of light through a photomask 135. The exposed photoresist 133 is subsequently developed and etched to form, as shown in FIG. 8E, the micro-pillars 137 on the first substrate 132, microdots 130, and micro-channel post 139. Several of the micro-pillars 137 intersect with each microdot 130 and micro-channel post 139.

Referring to FIG. 8F, the method of transferring the intersecting profiles of the microdots/micro-channel post and micro-pillars to a second substrate includes bringing a second substrate 140 into contact with the microdots 130, micro-channel post 139, and micro-pillars 137 on the first substrate 132. The second substrate 140 is pressed against the microdots 130 and micro-pillars 137 to make impressions of the intersecting microdots 130, micro-channel post 139, and micro-pillars 137 in the second substrate 140. These impressions become the micro-pillared structures and interconnect channel. Where the second substrate 140 is intended as the final product, the second substrate 140 is preferably made of a biocompatible material, as described with respect to the substrate of the cell culture apparatus above.

FIG. 8G shows the second substrate 140 with the micro-pillared structures 142 and interconnect channel 143, respectively. The process of making the cell culture apparatus may further include surface treating the micro-pillared structures 142. For example, a surface coating, such as collagen or other material desired to provide a specific microenvironment to be investigated, may be applied to the surfaces of the micro-pillars of the micro-pillared structures. To facilitate large-scale production of the cell culture apparatus, it may be desirable to form a replication molding tool. Referring to FIG. 8H, this may include, for example, growing metal 144 in the micro-pillared structures 142 and interconnect channel 143 and over the top surface of the second substrate 140 by processes such as electroplating. The grown metal is separated from the second substrate 140 and subsequently used as the replication molding tool, which has the negative copies of the desired micro-pillared structures and interconnect channel. FIG. 81 shows the replication molding tool 146.

For the cell culture apparatus including chambers above micro-pillared structure arrays, a substrate including the chambers can be formed using, for example, a photolithography process, such as described above, or any other suitable process for forming an array of trenches in a substrate, where the array of trenches serve as the array of chambers. Then, a replication molding tool formed as described above can be used to stamp the bottom of the chambers with the micro-pillared structures. Alternatively, an array of macro-pillars separated by trenches could be formed on a first substrate, where the macro-pillars would serve as negative copies of the chambers. Then, micro-posts could be formed on the macro-pillars. The micro-posts could be reshaped to microdots, which would serve as negative copies of the wells. A plurality of micro-pillars could be formed in intersecting relationship with the wells. Then the macro-pillars, wells, and micro-pillars can be impressed into a second substrate to form the cell culture apparatus. Alternatively, a substrate including orifices, which would serve as the chambers, may be formed and adjoined to a substrate including the micro-pillared structures. Any suitable process may be used to adjoin the substrates.

In one aspect, a cell culture apparatus 100 is provided for cultivating biological cells, e.g., a specific cell line. In another aspect, a cell culture apparatus 100 having micro-pillars is provided for cultivating biological cells or discovering optimum or effective cell-substrate interaction support structure for three-dimensional cell cluster growth. In the latter case, the cell culture apparatus 100 includes a wide variety of micro-pillared structures 105 with parameters spread over the micro-environment domain of interest.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A cell culture apparatus, comprising:
a substrate having a plurality of micro-wells wherein the micro-wells are formed from a plurality of micro-pillars.

2. The cell culture apparatus of claim 1, wherein at least one of the micro-wells is concave.

3. The cell culture apparatus of claim 1, wherein each micro-well has a diameter of from 50 to 400 microns.

4. The cell culture apparatus of claim 1, wherein each micro-well has a height of from 5 to 100 microns.

5. The cell culture apparatus of claim 1, wherein each micro-pillar has a diameter of from 2 to 20 microns.

6. The cell culture apparatus of claim 1, wherein a spacing between adjacent micro-pillars of from 5 to 100 microns.

7. The cell culture apparatus of claim 1, wherein at least two of the micro-wells have different topographies.

8. The cell culture apparatus of claim 1, further comprising at least one micro-channel formed in the substrate between adjacent micro-wells.

9. The cell culture apparatus of claim 1, wherein the micro-wells are grouped to form a plurality of micro-well arrays.

10. The cell culture apparatus of claim 9, wherein at least one of the micro-wells in a first one of the plurality of micro-well arrays has a topography which is different from a topography of at least one of the micro-wells in a second one of the plurality of micro-well arrays.

11. The cell culture apparatus of claim 9, further comprising at least one micro-channel formed in the substrate between adjacent micro-well arrays.

12. The cell culture apparatus of claim 9, wherein at least one of the plurality of micro-well arrays comprises micro-wells having uniform topography.

13. The cell culture apparatus of claim 9, wherein at least one of the plurality of micro-well arrays comprises micro-wells having non-uniform topography.

14. The cell culture apparatus of claim 1, wherein the micro-wells are open to a surface of the substrate.

15. The cell culture apparatus of claim 1, wherein the micro-wells are recessed relative to a surface of the substrate.

16. A method of making a cell culture apparatus, comprising:
forming a plurality of microdots on a first substrate, each microdot being a negative copy of a micro-well;
forming a plurality of micro-pillars on the plurality of microdots so that the micro-pillars abut the plurality of microdots, the number of micro-pillars being substantially larger than the number of microdots so that a plurality of the micro-pillars abut with each microdot; and
transferring abutting profiles of the microdots and micro-pillars to a second substrate to form a plurality of micro-pillared structures in the second substrate.

17. The method of claim 16, wherein each microdot is a negative copy of a concave well.

18. The method of claim 16, wherein forming a plurality of microdots comprises depositing a first photoresist on the first substrate, exposing the first photoresist to a pattern of light, and developing and etching the exposed first photoresist to form a plurality of micro-posts on the first substrate.

19. The method of claim 18, wherein forming a plurality of microdots further comprises shaping the micro-posts into the microdots by resist reflow.

20. The method of claim 18, wherein forming a plurality of micro-pillars on the plurality of microdots further comprises depositing a second photoresist on the microdots, exposing the second photoresist to a pattern of light, and developing and etching the exposed second photoresist to form the micro-pillars on the microdots.

21. The method of claim 16, wherein transferring abutting profiles of the microdots and micro-pillars to a second substrate comprises molding the second substrate around the intersecting microdots and micro-pillars.
